(19) United States
(12) Patent Application Publication
(10) Pub. No.: US 2026/0139241 A1
(43) Pub. Date: May 21, 2026

(54) METHOD FOR CONSTRUCTING SENESCENT CELLSAND METHOD FOR EVALUATING ANTI-SENESCENCE EFFICACY

(71) Applicant: JAKA BIOTECH Co,.Ltd., Shanghai (CN)

(72) Inventors: Apple ZHOU, Shanghai (CN); Inna LU, Shanghai (CN); Sera GAO, Shanghai (CN); Yasmine XIONG, Shanghai (CN); May SHI, Shanghai (CN)

(73) Assignee: JAKA BIOTECH Co,.Ltd., Shanghai (CN)

(21) Appl. No.: 18/857,906

(22) PCT Filed: May 11, 2023
(86) PCT No.: PCT/CN2023/093502

§ 371 Date: Oct. 18, 2024

(30) Foreign Application Priority Data
May 18, 2022 (CN) 202210563157.X

(51) Int. Cl. C12N 13/00; C12N 5/077; G01N 33/50

(52) U.S. Cl. CPC C12N 13/00; C12N 5/0656; G01N 33/502

(57) ABSTRACT

The present disclosure discloses a method for constructing senescent cells and a method for evaluating anti-aging efficacy. The method for constructing senescent cells comprises: applying inoculated culture and passage culture to primary cells and carrying out ultraviolet irradiation during the inoculated culture and passage culture to obtain UVA-P1 to UVA-Pn passage cells, and dividing various passages of cells into early-stage senescent cells, middle-stage senescent cells, and late-stage senescent cells according to detection results of the COL I content, MMP-1 content, and cell number of said various passages of cells. Said various passages of cells constructed in the present disclosure can be used to evaluate the anti-aging efficacy of skin. By means of correlating the constructed senescent cells at different stages with the skin senescence stage of populations of different ages, the correspondence is established between the passage number of the senescent cells and the age of the populations, such that the constructed senescent cells at different stages can be used as in vitro replacement cells for clinical senescence to carry out the corresponding anti-aging evaluation.

### TECHNICAL FIELD

The present disclosure pertains to the technical field of cell biology, and specifically relates to a method of constructing senescent cells and a method for evaluating anti-aging efficacy.

### BACKGROUND

As everyone is subject to aging from birth, skin aging is the most obvious and most intuitive manifestation of organismal aging and can be used as a predictor of life expectancy and health. Unlike other human organs, skin ages not only under inevitable influence of intrinsic factors, but under the impact of a variety of extrinsic environmental factors, which accelerate aging, especially ultraviolet radiation (UVR). Where, skin aging caused by environmental factors such as UV radiation, smoking, wind, sunshine, and harmful chemicals is called exogenous senescence. Among the environmental factors, the UV radiation in the sunlight is the main factor that contribute to skin aging. Thus, the exogenous senescence is also known as light aging. Both endogenous senescence and exogenous senescence can lead to reduction in the structural integrity of the skin, loss of function, and cumulative changes in appearance, such as more wrinkles, laxity, elastic fiber proliferation, angiotelectasis, and abnormal skin pigmentation. At present, although both of the skin aging processes not only are highly complex and incompletely understood, but also lack universal biomarkers for definitive detection and evaluation, the current studies agree that wrinkles are the external manifestation of skin aging, while internal cell senescence is the root cause.

Cell senescence is the prerequisite and ultimate driver of the physiological senescence process and is basically invisible during most of its progression. Cell senescence is a cell state that comes into being when stimulated by stress signals, is present in specific physiological processes, and links physiological and stress signals to intra-tissue homeostasis and tissue health. It can be triggered by a variety of different cell stresses, such as DNA damage, oncogene activation, oxidative stress, and exogenous toxic exposure. Cell senescence is not equivalent to cell death, in that senescent cells remain metabolically active for a period of time, still show some significant changes, and have four typical features: cell cycle arrest, senescence-associated secretory phenotypes, macromolecular damages, and metabolic disorders.

Currently, the main evaluation methods used for skin aging are clinical evaluation, recombinant three-dimensional dermis-epidermis skin model evaluation, and conventional in vitro cell evaluation.

The clinical evaluation has the advantage that it is closest to the real effect, while the disadvantages are as follows: 1) specialized evaluation equipment for clinical instruments are required, such as VISIA skin detector, skin microscope with an active skin surface analysis system Visioscan VC98, skin ultrasound detector, skin elasticity detector, and Antera 3D skin imaging measurement instrument; in general, the equipment are expensive and need to be operated by specialized personnel; 2) it is required that there should be at least three test members, one of whom is a clinician; 3) while it is required that the number of valid subjects should be at least 30 cases, it is difficult to recruit the subjects; moreover, different cosmetic products may be directed to different aging manifestations; 4) the experimental cycle of the clinical evaluation is relatively long, and it generally takes about 6 months from determining to carry out experiments to obtaining final results; 5) instead of throughput, only one sample generally can be tested at a time, which is not suitable for screening or testing raw materials of cosmetic products; 6) the cost of detection is relatively high; in general, the detection cost of one sample may be more than 100,000 RMB yuan; 7) the clinical evaluation is very large in error, and only when sample effects are extremely strong can relatively definitive results be obtained; 8) the clinical evaluation is high in safety risk.

The recombinant three-dimensional dermis-epidermis skin model evaluation has the advantage that it is relatively close to the clinical effect, while the disadvantages are as follows: 1) the cost of detection is relatively high; in general, the detection cost of one sample is at least 30,000 RMB yuan; 2) the experimental cycle is long; in general, a recombinant three-dimensional dermis-epidermis skin model is unavailable in a laboratory, and needs to be purchased; it generally takes at least 3 months from determining to carry out experiments to obtaining data results; 3) it cannot be used for high-throughput screening; 4) the aging period is not clear, while the most vigorous period of the recombinant three-dimensional dermis-epidermis skin model is often used to evaluate anti-aging effects of anti-aging products; moreover, the anti-aging effects of the products cannot be subdivided; 5) since the commercial recombinant dermis-epidermis skin model is relatively poor in stability at present, the trial-and-error cost is relatively high.

The conventional in vitro cell evaluation method has advantages as follows: 1) it can be used for high-throughput screening; 2) the experimental cycle is short; in general, it takes 7 working days from determining to carry out experiments to obtaining data results, while in the case of skilled operation, it may take 5 working days; 3) a few testing personnel are required: one sample generally requires one professional test member; 4) the detection is low in cost; in general, the detection cost of 10 samples is about 10,000 RMB yuan; 5) the error is the smallest; 6) the detection results are stable; when different personnel carry out tests in the same manner of treatment, the results are stable. However, the current in vitro cell evaluation method still has disadvantages as follows: 1) the correlation with clinical evaluation results is relatively low; 2) the aging period is not clear, and young cells may be used to evaluate anti-aging effects of anti-aging products; 3) as there is no clear subdivision into natural aging or light aging in vitro cell evaluation methods, the same sample may have different effects when evaluated in different manners of treatment.

### SUMMARY

In view of the defects in the prior art, it is an objective of the present disclosure to provide a method of constructing senescent cells and a method for evaluating anti-aging efficacy.

The objective of the present disclosure is achieved through the following technical solutions.

In a first aspect, the present disclosure provides a method for constructing exogenously senescent cells, including steps as follows:
A1, inoculating primary cells in a complete medium and culturing them for one week, applying medium change to the cells twice during the week, and applying UV irradiation to the cells in the course of each medium change, thereby obtaining UVA-P1 passage cells;
A2, carrying out the same treatment while replacing the primary cells in step A1 with said UVA-P1 passage cells, thereby obtaining UVA-P2 passage cells;
A3, repeating step A2, thereby obtaining corresponding UVA-P3 to UVA-Pn passage cells; where n in UVA-Pn is a positive integer from 4 to 20;
A4, applying detection of COL I content, MMP-1 content, and cell number to the UVA-P1 to UVA-Pn passage cells obtained in the preceding steps, and according to results of the detection, using said UVA-P3 to UVA-P4 passage cells as early-stage senescent cells, said UVA-P5 to UVA-P7 passage cells as middle-stage senescent cells, and said ≥UVA-P8 passage cells as late-stage senescent cells.

Preferably, in step A1, the primary cells are any one of human primary fibroblasts, human primary keratinocytes, and human primary melanocytes;
the primary cells have an inoculation density of 1.2×10⁵ to 6×10⁵/dish;
said cultivating is carried out under conditions of 37° C. and 5% CO₂.

Preferably, in step A1, specific steps of the medium exchange are: discarding a used medium, washing once with a PBS buffer solution, then adding the PBS buffer solution, pressing a UVA irradiator against a cell culture dish and irradiating it for 10 min, with an irradiation dose of 14.4 J; after finishing irradiating, discarding the PBS buffer solution, adding a fresh complete medium, and carrying on the culturing;
the medium change is carried out on the third and fifth days of the culturing.

Preferably, in step A4, the COL I content, MMP-1 content, and cell number of the early-stage senescent cells, middle-stage senescent cells, and late-stage senescent cells are shown in a table as follows:

| | Cell | COL I | MMP-1 | Cell |
|---|---|---|---|---|
| | Passage | Content | Content | Number × |
| | Number | pg/mL | pg/mL | 10⁶/dish |
| Early-Stage | UVA-P3 to | 3000 to 8000 | 2000 to 10000 | 9 to 9.8 |
| Senescent Cells | UVA-P4 | | | |
| Middle-Stage | UVA-P5 to | 600 to 3000 | 10000 to 20000 | 6 to 9 |
| Senescent Cells | UVA-P7 | | | |
| Late-Stage | ≥UVA-P8 | <600 | >20000 | <6 |
| Senescent Cells | | | | |

In a second aspect, the present disclosure provides a method for evaluating anti-aging efficacy of skin, including steps as follows:
S1, evaluating a clinically early-stage aging population aged 25 to 29 with said UVA-P3 to UVA-P4 passage cells prepared in the preceding method, evaluating a clinically middle-stage aging population aged 30 to 45 with said UVA-P5 to UVA-P7 passage cells, and evaluating a clinically late-stage aging population aged 46 and above with said ≥UVA-P8 passage cells;
S2, inoculating selected cells on a cell culture plate and culturing them for 24 h;
S3, discarding a used medium, adding a fresh complete medium with or without a sample to be detected to the cell culture plate, and carrying on the culturing for 24-96 h;
S4, detecting the cells cultured in step S3, using cells cultured without the sample to be detected as a control group, and evaluating the anti-aging efficacy of the sample to be detected.

Preferably, in step S2, the cells have an inoculation density of 5×10⁴ to 6×10⁵/well;
the cell culture plate is any one of a 6-well plate, a 12-well plate, a 24-well plate, a 48-well plate, and a 96-well plate;
in steps S2 and S3, said culturing is carried out under conditions of 37° C. and 5% CO₂.

Preferably, in step S4, when cultured cells are said UVA-P3 to UVA-P4 passage cells, detection indexes are cell cycle, DNA amount, and ATP;
when the cultured cells are said UVA-P5 to UVA-P7 passage cells, the detection indexes are COL I, MMP-1/-3, ATP, SA-β-gel, Hes-1, 8-OHdG, GLS1, and γH2AX;
when the cultured cells are said ≥UVA-P8 passage cells, the detection indexes are cell number, COL I, γH2AX, MMP-3, Hes-1, and 8-OHdG.

It is found in the preliminary experiments of the present disclosure that the use of different specific detection indexes based on cells at different senescence stages can make detection results more accurate.

In a third aspect, the present disclosure provides a method for evaluating anti-aging efficacy of an object to be detected on senescent cells at different stages, including steps as follows:
a. constructing endogenously senescent cells
a1. inoculating primary cells in a complete medium and culturing them for one week, applying medium change to the cells twice during the week, thereby obtaining P1 passage cells;
a2. carrying out the same treatment while replacing the primary cells in step a1 with said P1 passage cells, thereby obtaining P2 passage cells;
a3, repeating step a2, thereby obtaining corresponding P3 to Pn passage cells; where n is a positive integer from 11 to 20;
a4, applying detection of COL I content, MMP-1 content, and cell number to P1 to Pn passage cells obtained in the preceding steps, and according to results of the detection, using P3 to P4 passage cells as growth-stage senescent cells, P5 to P7 passage cells as early-stage senescent cells, P8 to P10 passage cells as middle-stage senescent cells, and ≥P11 passage cells as late-stage senescent cells.;
b. inoculating said P3 to P4 passage cells, said P5 to P7 passage cells, said P8 to P10 passage cells, and said ≥P11 passage cells prepared in step a and said UVA-P3 to UVA-P4 passage cells, said UVA-P5 to UVA-P7 passage cells, and said ≥UVA-P8 passage cells prepared in claim 1 on a cell culture plate respectively, and culturing them for 24 h;
c. discarding a used medium, adding a fresh complete medium with or without a sample to be detected to the cell culture plate, and carrying on the culturing for 24-96 h;
d. detecting cells cultured in step c, using cells cultured without the sample to be detected as a control group, and evaluating the anti-aging efficacy of the sample to be detected on each cell.

Preferably, in step a1, the primary cells have an inoculation density of 1.2×10⁵ to 6×10⁵/dish;
specific steps of the medium change are: discarding a used medium, washing once with a PBS buffer solution, then adding a fresh complete medium, and carrying on the culturing; the medium change is carried out on the third and fifth day of the culturing;
in step a4, the COL I content, MMP-1 content, and cell number of the early-stage senescent cells, middle-stage senescent cells, and late-stage senescent cells are shown in a table as follows:

| | Cell | COL I | MMP-1 | Cell |
|---|---|---|---|---|
| | Passage | Content | Content | Number × |
| | Number | pg/mL | pg/mL | 10⁶/dish |
| Growth-Stage | P3-P4 | >8000 | <2000 | 9.8 to 10 |
| Cells | | | | |
| Early-Stage | P5-P7 | 3000 to 8000 | 2000 to 10000 | 9 to 9.8 |
| Senescent Cells | | | | |
| Middle-Stage | P8-P10 | 600 to 3000 | 10000 to 20000 | 6 to 9 |
| Senescent Cells | | | | |
| Late-Stage | ≥P11 | <600 | >20000 | <6 |
| Senescent Cells | | | | |

In step b, the cells have an inoculation density of 5×10⁴ to 6×10⁵/well;
the cell culture plate is any one of a 6-well plate, a 12-well plate, a 24-well plate, a 48-well plate, and a 96-well plate;
in steps b and c, said culturing is carried out under conditions of 37° C. and 5% CO₂.

Preferably, in step d, when cultured cells are said UVA-P3 to UVA-P4 passage cells, detection indexes are cell cycle, DNA amount, and ATP;
when the cultured cells are said UVA-P5 to UVA-P7 passage cells, the detection indexes are COL I, MMP-1/-3, ATP, SA-β-gel, Hes-1, 8-OHdG, GLS1, and γH2AX;
when the cultured cells are said ≥UVA-P8 passage cells, the detection indexes are cell number, COL I, γH2AX, MMP-3, Hes-1, and 8-OHdG;
when the cultured cells are said P3 to P4 passage cells, the detection indexes are cell cycle, DNA amount, and ATP;
when the cultured cells are said P5 to P7 passage cells, the detection indexes are cell cycle, DNA amount, and ATP;
when the cultured cells are said P8 to P10 passage cells, the detection indexes are COL I, MMP-1/-3, ATP, SA-β-gel Hes-1, 8-OHdG, GLS1, and γH2AX;
when the cultured cells are said ≥P11 passage cells, the detection indexes are cell number, COL I, γH2AX, MMP-3, Hes-1, and 8-OHdG.

Compared with the prior art, the present disclosure has the following beneficial effects:
1. the present disclosure simulates exogenously senescent (light aging) cells by means of simultaneous occurrence of passage and ultraviolet (UVA) direct irradiation, and classifies the constructed senescent cells into early-stage senescent cells, middle-stage senescent cells, and late-stage senescent cells by detecting indexes of each senescent cell, and correlates the senescent cells at each stage with the skin senescence stages in which the populations of different ages are, so as to establish the correspondence between the passage number of the senescent cells and the age of the populations, and further use the constructed senescent cells at different stages as in vitro replacement cells for clinical senescence for the corresponding anti-aging evaluation. In this way, it provides an objective basis for the evaluation of the anti-aging efficacy of active ingredients of cosmetic products and the like against early-stage, middle-stage, and late-stage wrinkles among others, and offers a cytological method that can replace clinical practice with innovation and accuracy for tests of skin aging.
2. the results of the anti-aging efficacy evaluation from using the senescent cell model constructed in the present disclosure have a strong correlation with the results of the clinical evaluation, include fewer false-positive results, and possess the advantages of lower test cost, higher reproducibility, shorter test cycle, and higher throughput screening as compared with the method for the clinical evaluation;
3. the present disclosure further can accurately evaluate the effect of an object to be detected on endogenously or exogenously senescent cells at different stages by constructing exogenously (light aging) senescent cells and endogenously (naturally senescent) senescent cells at different stages, and provide an experimental basis for the accurate research and development of products with anti-aging efficacy.

### BRIEF DESCRIPTION OF THE DRAWINGS

Other features, objectives, and advantages of the present disclosure will become more apparent by reading the detailed description of the nonrestrictive embodiments with reference to the following drawings:

FIG. 1 shows detection results of endogenously senescent cells and exogenously senescent cells of different passages as constructed in Embodiments 1 and 2; where, FIG. 1A shows results of COL I content; FIG. 1B shows results of MMP-1 content; and FIG. 1C shows results of the cell number; where Group Aging represents the endogenously senescent cells and Group UVA-Aging represents the exogenously senescent cells;

FIG. 2 shows proliferation-promoting effects of senescent cells at different stages in Embodiment 3;

FIG. 3 shows results from detecting changes in high echo areas of dermis layers of populations of different ages by using a skin ultrasound detector in Embodiment 4;

FIG. 4 shows results of evaluation on the mitochondrial membrane integrity in a model of senescent cells at different stages in Embodiment 4;

FIG. 5 shows SA-β-SA evaluation results of a model of senescent cells at different stages in Embodiment 4;

FIG. 6 shows results of changes in high echo areas of dermis layers of a clinical middle-stage skin senescence model where a Biomenta sample at a concentration of 3% is used in Embodiment 4.

### DETAILED DESCRIPTION

The present disclosure is described in detail below in combination with specific embodiments. The following embodiments will help those skilled in the art to further understand the present disclosure, but do not limit the present disclosure in any way. It should be noted that those skilled in the art can make several transformations and improvements without departing from the conception of the present disclosure. These all fall within the scope of protection of the present disclosure.

Such words as “preferable”, “preferably”, and “more preferable” in the present disclosure refer to embodiments of the present disclosure, which can provide certain beneficial effects in certain cases. However, other embodiments may also be preferrable in the same or other cases. Furthermore, the expression of one or more preferred embodiments does not imply that other embodiments are not feasible, nor is it intended to exclude other embodiments from the scope of the present disclosure.

As should be understood, all numbers that denote, e.g., the amount of ingredients used in the description and claims should be understood to be modified by the term “about” in all cases, other than in any instance of operation or in cases indicated otherwise. Thus, unless the opposite is stated, the numerical parameters set forth in the following description and appended claims are approximate values that vary according to desired properties to be obtained by the present disclosure. At least instead of attempting to confine the application of the principle of equivalence to the scope of the claims, each numerical parameter should at least be interpreted in light of the number of valid numbers as reported and by means of applying ordinary rounding techniques.

Although the numerical ranges and parameters that set forth the broad scope of the present disclosure are approximate values, the values listed in the specific instances are reported as accurately as possible. However, any numerical value inherently contains certain errors that necessarily arise from standard deviations found in tests and measurements of their own.

Fibroblasts are the most abundant cell type in the dermis, contribute directly or indirectly to skin aging through interactions with other cells, and are frequently used in in vitro studies of skin aging. Human fibroblasts are functionally active cells; both the cells and nuclei thereof are relatively large and clearly outlined; the nuclei are large and distinct; the cytoplasm is weakly basophilic, and has significant activities of synthesis and secretion of protein. The present disclosure establishes an in vitro human primary fibroblast senescence model by studying common points of human primary fibroblasts in natural senescence and light senescence in terms of cell cycle, senescence-related secretion phenotypes, macromolecular damages, and metabolic disorders among others. At the same time, the correlation between in vitro human primary fibroblast senescence and clinical senescence is established by comparing some cell senescence indexes with human clinical senescence indexes. In this method, the results of in vitro evaluation are improved to get closer to the results of clinical evaluation, which provides an objective basis for the evaluation of active ingredients of cosmetic products and the like on senescence.

Fibroblast senescence is closely relevant to the development of skin aging. Fibroblasts are the leading cells that constitute the dermis layer of skin, and the dysfunction thereof is closely relevant to most of changes in the skin phenotype, such as formation of wrinkles. Thus, dermal fibroblasts become the preferred model for studying endogenous and exogenous senescence at the cellular level. The human primary fibroblasts used in the following embodiments were isolated from human prepuce tissues by the present laboratory, with donor information: male, 20 years old. Cell identification was based on functional realizations of fibroblast collagen/MMP-1/MMP-3 among others as well as transcriptome gene comparisons. The cell number is G2018001FF.

The complete medium for fibroblasts as used in the following embodiments contains 10% new-born calf serum, 1% mixed solution of three, i.e., penicillin, streptomycin, and amphotericin B, and a DMEM basal medium as balance.

### Embodiment 1

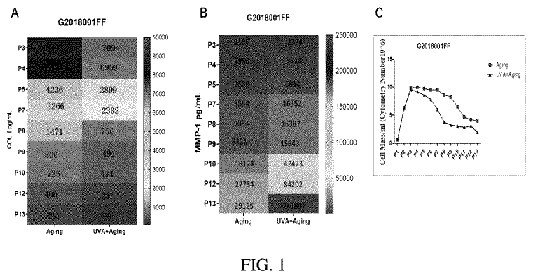

This embodiment provides a method for constructing an endogenously senescent (naturally senescent) cell model, and specific steps are as follows:
1) taking isolated human primary fibroblasts No. G2018001FF, inoculating them at an inoculation density of 7.5×10⁵/T175 vial in a T175 vial containing a complete medium for fibroblasts, culturing them for 7 days, and labeling obtained cells as P0 passage cells;
2) on the first day, inoculating P0 passage cells at a density of 6×10⁵/dish in a 10 cm cell culture dish, culturing them, and labeling obtained cells as P1 passage cells, where the medium as used is a complete medium for fibroblasts;
3) on the third and fifth days, applying medium change to said P1 passage cells, where specific operations are as follows: discarding a used medium, washing once with a PBS buffer solution (a phosphate buffer solution), adding a fresh complete medium for fibroblasts, and placing under conditions of 37° C. and 5% CO₂ for passage culture;
4) on the eighth day, applying cell digestion and counting to said P1 passage cells, thereby obtaining said P1 passage cells and cell number thereof; where specific operations of said cell digestion and counting are as follows: discarding a used medium, washing twice with a PBS buffer solution, adding 2 mL of 0.05% trypsin digestive fluid per dish, digesting for 3 min at 37° C., discarding the trypsin digestive fluid, then terminating the digestion by adding 5 mL of complete medium to each dish, gently pipetting the cells, thereby obtaining cell suspension; making measurement 8 times directly with a cell counter, and averaging the cell number (see Table 1);
5) on the eighth day, executing the operations in steps 2) to 4) on said P1 passage cells obtained after the above passage culture, thereby obtaining P2 passage cells; repeating the practice, thereby obtaining P3 to P14 passage cells;
6) inoculating P1 to P14 passage cells into a 96-well cell culture plate at 2×10⁴/well, and culturing them in an incubator under conditions of 37° C. and 5% CO₂ for 24 h, where the medium as used is the above complete medium for fibroblasts; applying medium change to the cells obtained by culturing, where specific operations are as follows: after discarding the medium, adding a fresh complete medium for fibroblasts to the culture plate, and culturing in an incubator under conditions of 37° C. and 5% CO₂ for 48 h; after finishing culturing, collecting each medium supernatant, and completing detection by using Collagen I (COL I) and MMP-1 Elisa kits according to the standard operation procedures of the Elisa kit, thereby obtaining results of COL I content in each passage of cells (as shown by Group Aging in FIG. 1A) and results of MMP-1 (as shown by Group Aging in FIG. 1B); counting each passage of cells in the method in step 4), thereby obtaining the cell number (as shown by Group Aging in FIG. 1C);
7) based on the results of each detection in step 6), classifying said P1 to P14 passage cells into growth-stage cells (P3 to P4 passage cells), early-stage senescent cells (P5 to P7 passage cells), middle-stage senescent cells (P8 to P10 passage cells), and late-stage senescent cells (P10 to P14 passage cells) according to the ranges as shown in Table 1.

TABLE 1

| | COL I Content | MMP-1 Content | Cell Number × |
|---|---|---|---|
| | pg/mL | pg/mL | 10⁶/dish |
| Growth-Stage | >8000 | <2000 | 9.8 to 10 |
| Cells | | | |
| Early-Stage | 3000 to 8000 | 2000 to 10000 | 9 to 9.8 |
| Senescent Cells | | | |
| Middle-Stage | 600 to 3000 | 10000 to 20000 | 6 to 9 |
| Senescent Cells | | | |
| Late-Stage | <600 | >20000 | <6 |
| Senescent Cells | | | |

### Embodiment 2

This embodiment provides a method for constructing an exogenously senescent (light aging) cell model, and specific steps are as follows:
1) taking human primary fibroblasts No. G2018001FF (hereinafter referred to as P0 passage cells);
2) on the first day, inoculating P0 passage cells at a density of 6×10⁵/dish in a 10 cm cell culture dish, culturing them, and labeling obtained cells as UVA-P1 passage cells, where the medium as used is a complete medium for fibroblasts, which medium contains 10% new-born calf serum, 1% mixed solution of three, i.e., penicillin, streptomycin, and amphotericin B, and a DMEM basal medium as balance;
3) on the third and fifth days, applying medium change to said UVA-P1 passage cells, where specific operations are as follows: discarding a used medium, washing once with a PBS buffer solution, adding 5 ml of PBS buffer solution, pressing a central portion of a UVA irradiator against a cell culture dish and irradiating it for 10 min (with an irradiation dose of 14.4 J); then, discarding the PBS buffer solution, adding a fresh complete medium for fibroblasts, and placing under conditions of 37° C. and 5% CO₂ for passage culture;
4) on the eighth day, applying cell digestion and counting to said UVA-P1 passage cells, thereby obtaining said UVA-P1 passage cells and cell number thereof; where specific operations of said cell digestion and counting are as follows: discarding a used medium, washing twice with a PBS buffer solution, adding 2 mL of 0.05% trypsin digestive fluid per dish, digesting for 3 min at 37° C., discarding the trypsin digestive fluid, then terminating the digestion by adding 5 mL of complete medium to each dish, gently pipetting the cells, thereby obtaining cell suspension; making measurement 8 times directly with a cell counter, and averaging the cell number (see Table 2);
5) on the eighth day, executing the operations in steps 2) to 4) on said UVA-P1 passage cells obtained after the above passage culture, thereby obtaining UVA-P2 passage cells; repeating the practice, thereby obtaining UVA-P3 to UVA-P14 passage cells.
6) inoculating UVA-P1 to UVA-P14 passage cells into a 96-well cell culture plate at 2×10⁴/well, and culturing them in an incubator under conditions of 37° C. and 5% CO₂ for 24 h, where the medium as used is the above complete medium for fibroblasts; applying medium change to the cells obtained by culturing, where specific operations are as follows: after discarding the medium, adding a fresh complete medium for fibroblasts to the culture plate, and culturing in an incubator under conditions of 37° C. and 5% CO₂ for 48 h; after finishing culturing, collecting each medium supernatant, and completing detection by using Collagen I (COL I) and MMP-1 Elisa kits according to the standard operation procedures of the Elisa kit, thereby obtaining results of COL I content in each passage of cells (as shown by Group UVA-Aging in FIG. 1A) and results of MMP-1 (as shown by Group UVA-Aging in FIG. 1B); counting each passage of cells in the method in step 4), thereby obtaining the cell number (as shown by Group UVA-Aging in FIG. 1C);
7) based on the results of each detection in step 6), classifying said UVA-P1 to UVA-P14 passage cells into early-stage senescent cells (UVA-P3 to UVA-P4 passage cells), middle-stage senescent cells (UVA-P5 to UVA-P7 passage cells), and late-stage senescent cells (UVA-P8 to P14 passage cells) according to the ranges as shown in Table 2.

TABLE 2

| | COL I Content | MMP-1 Content | Cell Number × |
|---|---|---|---|
| | pg/mL | pg/mL | 10⁶/dish |
| Early-Stage | 3000 to 8000 | 2000 to 10000 | 9 to 9.8 |
| Senescent Cells | | | |
| Middle-Stage | 600 to 3000 | 10000 to 20000 | 6 to 9 |
| Senescent Cells | | | |
| Late-Stage | <600 | >20000 | <6 |
| Senescent Cells | | | |

### Embodiment 3

This embodiment provides a method for evaluating anti-aging efficacy of an object to be detected on senescent cells at different stages, which method has VC as a test sample, and includes the following steps:
1) taking P4 passage cells (growth-stage cells), P7 passage cells (early-stage senescent cells), P9 passage cells (middle-stage senescent cells), and P12 passage cells (late-stage senescent cells) prepared in Embodiment 1, and UVA-P4 passage cells (early-stage senescent cells), UVA-P7 passage cells (middle-stage senescent cells), and UVA-P9 passage cells (late-stage senescent cells) prepared in Embodiment 2, inoculating each of them on a 96-well cell culture plate (or a 60 mm cell culture dish) at 2×10⁴/well (or 0.8×10⁵/dish), and culturing in an incubator under conditions of 37° C. and 5% CO₂ for 24 h, where the culture medium as used is a complete medium for fibroblasts;
2) after finishing culturing in step 1), discarding a used medium, adding a fresh medium for detection without a test sample to the culture plate as a control group, adding a fresh medium for detection with a test sample (10 ug/mL VC) as an experimental group, and carrying on the culturing in an incubator under conditions of 37° C. and 5% CO₂ for 48 h, where the medium for detection contains 1% new-born calf serum, 1% mixed solution of three, i.e., penicillin, streptomycin, and amphotericin B, and a DMEM basal medium as balance;
3) after finishing culturing in step 2), applying detection of corresponding indexes to senescent cells at each stage, where each cell detection index is specifically shown in Table 3. As collected supernatant is used, COL I and MMP-1 Elisa kits can be used to complete the detection according to the standard operation procedures of the Elisa kit; cells inoculated in 96-well plates can be used to detect ATP according to the standard operation procedures of the ATP kit, the specific MitoBright LT fluorescent probe can be used to detect the mitochondrial membrane integrity according to the standard operation procedures of this kit, DNA amount can be detected according to the standard procedures of Hoechest33342 kit, METTL3, GLS1, 8-OHdG, γH2AX, Hes-1 can be detected according to the standard procedures of immunofluorescence, and SA-β-gel can be detected according to the standard procedures of SA-β-gel kit; cells inoculated in 60 mm cell culture dishes can be subjected to cell cycle detection according to the standard operation procedures of cell cycle detection with flow cytometry.

TABLE 3

| | Cell | | | |
|---|---|---|---|---|
| | Senescence | Endogenous | Exogenous | |
| | Stage | Senescence | Senescence | Detection Index |
| 1 | Growth- | P3-P4 | / | Cell cycle, DNA amount, |
| | Stage Cells | | | ATP |
| 2 | Early-Stage | P5-P7 | UVA-P3 to | Cell cycle, DNA amount, |
| | Senescent | | UVA-P4 | ATP |
| | Cells | | | |
| 3 | Middle- | P8-P10 | UVA-P5 to | COL I, MMP-1/-3, ATP, |
| | Stage | | UVA-P7 | SA-β-gel, Hes-1, |
| | Senescent | | | 8-OHdG, GLS1, γH2AX |
| | Cells | | | |
| 4 | Late-Stage | ≥P11 | ≥UVA-P8 | Cell number, COL I, |
| | Senescent | | | γH2AX, MMP-3, Hes-1, |
| | Cells | | | 8-OHdG |

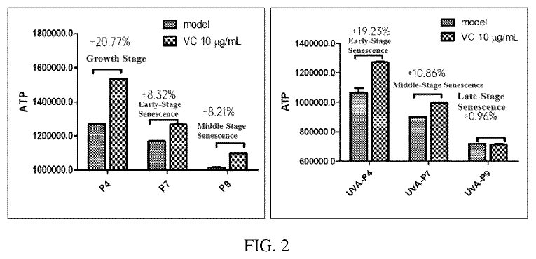

The detection results show that for endogenous senescence (natural senescence), VC has a relatively significant proliferation-promoting effect on growth-stage cells, a relatively poor proliferation-promoting effect on early-stage senescent cells, and basically no proliferation-promoting effect on middle-stage senescent cells and late-stage senescent cells. For exogenous senescence (light aging), VC has a relatively significant proliferation-promoting effect on early-stage senescent cells, a relatively poor proliferation-promoting effect on middle-stage senescent cells, and no proliferation-promoting effect on late-stage senescent cells. FIG. 2 shows results of ATP detection on P4 passage cells, P7 passage cells, UVA-P4 passage cells, and UVA-P7 passage cells with or without VC.

### Embodiment 4

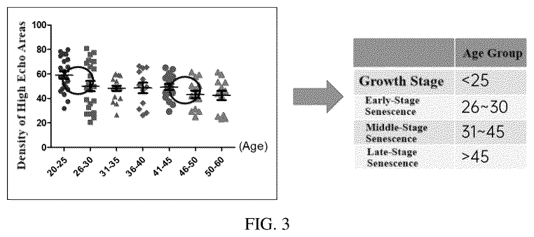

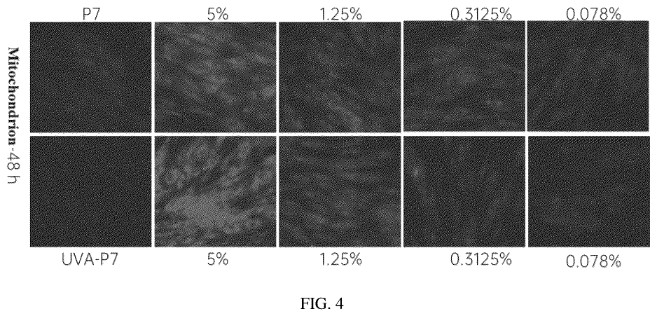

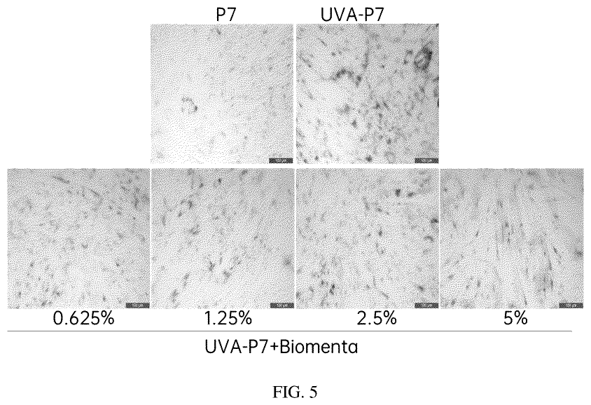

This embodiment provides a method for evaluating anti-aging efficacy of an object to be detected on senescent cells at different stages, which method has Biomenta as a test sample (produced by Shanghai Jiakai Biotechnology Co. Ltd. and used as a lysate product of Bifidobacterium adolescentis strains obtained in the method in the patent document CN114292773A), and includes the following steps:
1) using P7 passage cells (early-stage senescent cells) and P9 passage cells (middle-stage senescent cells) prepared in Embodiment 1 and UVA-P4 passage cells (early-stage senescent cells) and UVA-P7 passage cells (middle-stage senescent cells) prepared in Embodiment 2, inoculating each of them at 2×10⁴/well (or 0.8×10⁵/dish) on a 96-well cell culture plate (or a 60 mm cell culture dish), and culturing in an incubator under conditions of 37° C. and 5% CO₂ for 24 h, where the medium as used is a complete medium for fibroblasts;
2) after 24 h, discarding a used medium, and adding a fresh medium for detection with or without Biomenta to the culture plate, and carrying on the culturing in the incubator under conditions of 37° C. and 5% CO₂ for 48 h; where, after finishing culturing, the mitochondrial membrane integrity is detected according to the standard procedures of a mitochondrion detection kit to evaluate the function of mitochondria; SA-β-SA is detected according to the standard procedures of a SA-β-SA detection kit to evaluate the cell senescence; as part of the results of the mitochondrion detection (P7 passage cells and UVA-P7 passage cells) are shown in FIG. 4, Biomenta at a volumetric concentration of 0.078% to 5% has a certain inhibiting effect on both early-stage senescent cells and middle-stage senescent cells; where the inhibiting effect of Biomenta on the middle-stage senescent cells is greater than that on the early-stage senescent cells, and the effect of Biomenta on middle-stage exogenously senescent (light aging) cells is more significant than that on middle-stage endogenously senescent (naturally senescent) cells; as part of the results of SA-β-SA detection (P7 passage cells and UVA-P7 passage cells) is shown in FIG. 5, Biomenta at a volumetric concentration of 0.625% to 5% has a certain inhibiting effect on middle-stage senescent cells, but Biomenta has no significant effect on early-stage senescent cells, and the effect of Biomenta on middle-stage exogenously senescent (light aging) cells is more significant than that on middle-stage endogenously senescent (naturally senescent) cells;
3) detecting anti-aging clinical effects by using Biomenta, where specific operations are as follows:
3.1 the test method was approved by the Ethics Committee, and volunteers signed an informed consent form;
3.2 the age of the volunteers ranged from 18 years old to 65 years old;
3.3 the skin data of the volunteers were collected by a skin ultrasound detector, and according to changes in high echo areas (collagen) of dermis layers of populations of different ages (as shown in FIG. 3), the clinical skin was classified into early-stage senescence, middle-stage senescence, and late-stage senescence, and one-to-one correspondence was established with the passage number of cells at the early, middle, and late stages of cell senescence, as shown in Table 3.

TABLE 3

| | | | | Age |
|---|---|---|---|---|
| | Exogenously | Endogenously | | Group |
| | Senescent | Senescent | Clinical | (Years |
| | Cell Passage | Cell Passage | Skin | Old) |
| Cell | P3-P4 | / | Growth Stage | <25 |
| Senescence | | | | |
| Stage | | | | |
| Growth-Stage | P5-P7 | P3-P4 | Early-Stage | 25-29 |
| Cells | | | Senescence | |
| Early-Stage | P8-P10 | P5-P7 | Middle-Stage | 30-45 |
| Senescent | | | Senescence | |
| Cells | | | | |
| Middle-Stage | ≥P11 | ≥P8 | Late-Stage | >45 |
| Senescent | | | Senescence | |
| Cells | | | | |
| Late-Stage | | | | |
| Senescent | | | | |
| Cells | | | | |

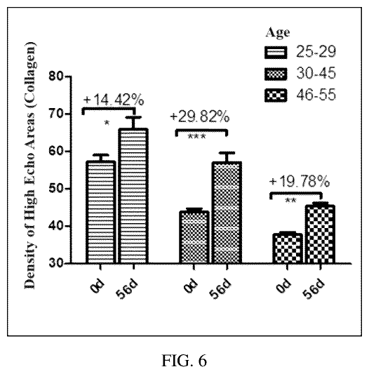

3.4 Volunteers aged 25-55 acted as test objects; according to standard procedures of the clinical evaluation, a skin ultrasound detector (a Danish DermaLab Combo skin ultrasound detector) was used to detect the density of high echo areas (collagen) of dermis layers before applying a Biomenta sample at a concentration of 3% to the volunteers (0 d) and after 56 days (56 d) of consecutively applying the Biomenta sample at a concentration of 3%. The results are shown in FIG. 6: Biomenta at a concentration of 3% has the most significant mitigating effect on middle-stage senescent skin (volunteers aged 30-45). It is consistent with the results of middle-stage exogenously senescent (light aging) cells in step 2).

The results of this embodiment indicate that UVA-P3\UVA-P4 passage cells obtained in the method of Embodiment 2 can be used as in vitro replacement cells for clinical early-stage senescence (26-30 years old) to carry out early-stage senescence evaluation, UVA-P5 to UVA-P7 passage cells can be used as in vitro replacement cells for clinical middle-stage senescence (31-45 years old) to carry out middle-stage senescence evaluation, and UVA-P8 or greater can be used as in vitro replacement cells for clinical late-stage senescence (46 years old or older) to carry out late-stage senescence evaluation.

There are many specific approaches to application of the present disclosure, and those described above are only preferred embodiments of the present disclosure. It should be noted that the above embodiments are only used to illustrate the present disclosure, and are not intended to limit the scope of protection of the present disclosure. Without departing from the principles of the present disclosure, those skilled in the art can make a number of improvements, which should also be deemed to fall within the scope of protection of the present disclosure.

1. A method for constructing exogenously senescent cells, comprising steps as follows:
A1, inoculating primary cells in a complete medium and culturing them for one week, applying medium change to the cells twice during the week, and applying UV irradiation to the cells in the course of each medium change, thereby obtaining UVA-P1 passage cells;
A2, carrying out the same treatment while replacing the primary cells in step A1 with said UVA-P1 passage cells, thereby obtaining UVA-P2 passage cells;
A3, repeating step A2, thereby obtaining corresponding UVA-P3 to UVA-Pn passage cells; wherein n in UVA-Pn is a positive integer from 4 to 20;
A4, applying detection of COL I content, MMP-1 content, and cell number to UVA-P1 to UVA-Pn passage cells obtained in the preceding steps, and according to results of the detection, using UVA-P3 to UVA-P4 passage cells as early-stage senescent cells, UVA-P5 to UVA-P7 passage cells as middle-stage senescent cells, and ≥UVA-P8 passage cells as late-stage senescent cells.

2. The method for constructing exogenously senescent cells according to claim 1, wherein in step A1, the primary cells are any one of human primary fibroblasts, human primary keratinocytes, and human primary melanocytes;
the primary cells have an inoculation density of 1.2×10⁵ to 6×10⁵/dish;
said culturing is carried out under conditions of 37° C. and 5% CO₂.

3. The method for constructing exogenously senescent cells according to claim 1, wherein in step A1, specific steps of the medium exchange are: discarding a used medium, washing once with a PBS buffer solution, then adding the PBS buffer solution, pressing a UVA irradiator against a cell culture dish and irradiating it for 10 min, with an irradiation dose of 14.4 J; after finishing irradiating, discarding the PBS buffer solution, adding a fresh complete medium, and carrying on the culturing;
the medium change is carried out on the third and fifth days of the culturing.

4. The method for constructing exogenously senescent cells according to claim 1, wherein in step A4, COL I contents, MMP-1 contents, and cell number of the early-stage senescent cells, middle-stage senescent cells, and late-stage senescent cells are shown in a table as follows:

| | Cell | COL I | MMP-1 | Cell |
|---|---|---|---|---|
| | Passage | Content | Content | Number × |
| | Number | pg/mL | pg/mL | 10⁶/dish |
| Early-Stage | UVA-P3 to | 3000~8000 | 2000~10000 | 9~9.8 |
| Senescent Cells | UVA-P4 | | | |
| Middle-Stage | UVA-P5 to | 600~3000 | 10000~20000 | 6~9 |
| Senescent Cells | UVA-P7 | | | |
| Late-Stage | ≥UVA-P8 | <600 | >20000 | <6 |
| Senescent Cells | | | | |

5. A method for evaluating anti-aging efficacy of skin, comprising steps as follows:
S1, evaluating a clinically early-stage aging population aged 25 to 29 with said UVA-P3 to UVA-P4 passage cells prepared in the method of claim 1, evaluating a clinically middle-stage aging population aged 30 to 45 with said UVA-P5 to UVA-P7 passage cells, and evaluating a clinically late-stage aging population aged 46 and above with said ≥UVA-P8 passage cells;
S2, inoculating selected cells on a cell culture plate and culturing them for 24 h;
S3, discarding a used medium, adding a fresh complete medium with or without a sample to be detected to the cell culture plate, and carrying on the culturing for 24-96 h;
S4, detecting the cells cultured in step S3, using cells cultured without the sample to be detected as a control group, and evaluating the anti-aging efficacy of the sample to be detected.

6. The method for evaluating anti-aging efficacy of skin according to claim 5, wherein in step S2, the cells have an inoculation density of 5×10⁴ to 6×10⁵/well;
the cell culture plate is any one of a 6-well plate, a 12-well plate, a 24-well plate, a 48-well plate, and a 96-well plate;
in steps S2 and S3, said culturing is carried out under conditions of 37° C. and 5% CO₂.

7. The method for evaluating anti-aging efficacy of skin according to claim 5, wherein in step S4, in case that cultured cells are said UVA-P3 to UVA-P4 passage cells, detection indexes are cell cycle, DNA amount, and ATP;
in case that the cultured cells are said UVA-P5 to UVA-P7 passage cells, the detection indexes are COL I, MMP-1/-3, ATP, SA-β-gel, Hes-1, 8-OHdG, GLS1, and γH2AX;
in case that the cultured cells are said ≥UVA-P8 passage cells, the detection indexes are cell number, COL I, γH2AX, MMP-3, Hes-1, and 8-OHdG.

8. A method for evaluating anti-aging efficacy of an object to be detected on senescent cells at different stages, comprising steps as follows:
a. constructing endogenously senescent cells:
a1. inoculating primary cells in a complete medium and culturing them for one week, and applying medium change to the cells twice during the week, thereby obtaining P1 passage cells;
a2. carrying out the same treatment while replacing the primary cells in step a1 with said P1 passage cells, thereby obtaining P2 passage cells;
a3, repeating step a2, thereby obtaining corresponding P3 to Pn passage cells; wherein n is a positive integer from 11 to 20;
a4, applying detection of COL I content, MMP-1 content, and cell number to P1 to Pn passage cells obtained in the preceding steps, and according to results of the detection, using P3 to P4 passage cells as growth-stage senescent cells, P5 to P7 passage cells as early-stage senescent cells, P8 to P10 passage cells as middle-stage senescent cells, and ≥P11 passage cells as late-stage senescent cells;
b. inoculating said P3 to P4 passage cells, said P5 to P7 passage cells, said P8 to P10 passage cells, and said ≥P11 passage cells prepared in step a and said UVA-P3 to UVA-P4 passage cells, said UVA-P5 to UVA-P7 passage cells, and said ≥UVA-P8 passage cells prepared in claim 1 on a cell culture plate respectively, and culturing them for 24 h;
c. discarding a used medium, adding a fresh complete medium with or without a sample to be detected to the cell culture plate, and carrying on the culturing for 24-96 h;
d. detecting cells cultured in step c, using cells cultured without the sample to be detected as a control group, and evaluating the anti-aging efficacy of the sample to be detected on each cell.

9. The method for evaluating anti-aging efficacy of an object to be detected on senescent cells at different stages according to claim 8, wherein in step a1, the primary cells have an inoculation density of 1.2×10⁵ to 6×10⁵/dish;
specific steps of the medium change are: discarding the used medium, washing once with a PBS buffer solution, then adding a fresh complete medium, and carrying on the culturing; the medium change is carried out on the third and fifth day of the culturing;
in step b, the cells have an inoculation density of 5×10⁴ to 6×10⁵/well;
the cell culture plate is any one of a 6-well plate, a 12-well plate, a 24-well plate, a 48-well plate, and a 96-well plate;
in steps b and c, said culturing is carried out under conditions of 37° C. and 5% CO₂.

10. The method for evaluating anti-aging efficacy of an object to be detected on senescent cells at different stages according to claim 8, wherein in step d, in case that cultured cells are said UVA-P3 to UVA-P4 passage cells, detection indexes are cell cycle, DNA amount, and ATP;
in case that the cultured cells are said UVA-P5 to UVA-P7 passage cells, the detection indexes are COL I, MMP-1/-3, ATP, SA-β-gel, Hes-1, 8-OHdG, GLS1, and γH2AX;
in case that the cultured cells are said ≥UVA-P8 passage cells, the detection indexes are cell number, COL I, γH2AX, MMP-3, Hes-1, and 8-OHdG;
in case that the cultured cells are said P3 to P4 passage cells, the detection indexes are cell cycle, DNA amount, and ATP;
in case that the cultured cells are said P5 to P7 passage cells, the detection indexes are cell cycle, DNA amount, and ATP;
in case that the cultured cells are said P8 to P10 passage cells, the detection indexes are COL I, MMP-1/-3, ATP, SA-β-gel Hes-1, 8-OHdG, GLS1, and γH2AX;
in case that the cultured cells are said ≥P11 passage cells, the detection indexes are cell number, COL I, γH2AX, MMP-3, Hes-1, and 8-OHdG.
